(19) United States
(12) Patent Application Publication
(10) Pub. No.: US 2025/0389662 A1
(43) Pub. Date: Dec. 25, 2025

(54) ILLUMINATION AND IMAGING SYSTEM IN TDI-BASED CONTINUOUS LINE SCANNING MICROSCOPY

(71) Applicant: Miltenyi Biotec, Inc., Gaithersburg, MD (US)

(72) Inventors: Robin DIEKMANN, Bergisch Gladbach (DE); Heinrich SPIECKER, Bergisch Gladbach (DE); Andy SISCHKA, Bergisch Gladbach (DE); Chris NEHME, Bergisch Gladbach (DE); Hsinyi SMITH, Bergisch Gladbach (DE); Hansueli MEYER, Gaithersburg, MD (US)

(73) Assignee: Miltenyi Biotec, Inc., Gaithersburg, MD (US)

(21) Appl. No.: 18/879,414

(22) PCT Filed: Jun. 27, 2023
(86) PCT No.: PCT/US2023/026298

§ 371 Date: Dec. 27, 2024

Related U.S. Application Data
(60) Provisional application No. 63/357,737, filed on Jul. 1, 2022.

(51) Int. Cl. G01N 21/64

(52) U.S. Cl. CPC G01N 21/6458; G01N 21/6408; G01N 21/6486; G01N 2021/6471

(57) ABSTRACT

An imaging system for imaging a biological sample or another sample containing fluorescent molecules may include an optical system with a light source emitting light, wherein the light is directed by the optical system to the sample via at least one plane being conjugate to the image plane. The optical source may have an extended radiation pattern, in other words, the radiation beam may have an extent in the x-and y-planes, rather than a point source. The extent of the illumination region in x and y may be based on and matched to the detector area onto which the radiation may be imaged, preferably a TDI detector. This novel system may include genomics, proteomics and transcriptomics work flows.

### CROSS REFERENCE TO RELATED APPLICATIONS

This US non-Provisional Patent Application claims priority to U.S. Provisional Application Ser. No. 63/357,737 (the '737 application), filed on Jul. 1, 2022. The '737 application is incorporated by reference in its entirety.

### STATEMENT REGARDING FEDERALLY SPONSORED RESEARCH

Not applicable.

### STATEMENT REGARDING MICROFICHE APPENDIX

Not applicable.

### BACKGROUND

The present invention relates to time delayed integration (TDI) microscopy for optical imaging of biological samples.

A continuous line scanning microscope based on time-delayed-integration (TDI) camera detection offers significantly higher throughput compared to traditional stop- and-stare approaches. The TDI-based detection is therefore a favorable candidate for various existing imaging systems, as well as for the next generation biological imaging systems, that require high-throughput imaging, where optionally additional optical sectioning and ability to image volume samples such as tissue sections, organoids or whole organs are highly desired. To enable high-throughput microscopy, high illumination intensities are required. Though high intensity illumination can be introduced by illumination with a single diffraction-limited point focus or line focus, this approach comes with many disadvantages since expensive and bulky laser instrumentation is required and scanning speed is either restricted by low fluorescence, luminescence, or phosphorescence signal from the small illuminated area, and/or local intensities have to be extremely high, potentially leading to sample damage.

One application in which speed is particularly helpful is genetic sequencing. Throughput in next generation sequencing (NGS) by synthesis as well as spatial multi-omics approaches (e.g. spatial proteomics, spatial transcriptomics, spatial genomics or a combination thereof) rely on the speed of the optical microscope. A continuous stage scanning concept together with detection by a multi-channel TDI camera setup was developed, requiring multi-color, high power, flat line, sharp edge illumination to the sample. For most efficient use of available light power, the line aspect ratio has to be up to x:y=32:1 or higher (depending on the camera sensor geometry) at a line width of 1 mm or more while not exceeding a line height of 32 μm. These numbers are examples for a TDI camera featuring a detector array of 128 pixels in scan direction and 4000 pixels perpendicular to the scan direction, a pixel width and height of 5 μm, respectively, and a microscope that has a 20× magnification. Additionally, following features of the illumination system are desired: multi-color excitation, spectrally separated excitation for suppression of cross-talk between fluorescent channels, cost efficiency, modularity, low maintenance effort, to name a few.

### SUMMARY OF THE INVENTION

To overcome the illumination issue while still maintaining the advantages of TDI-based detection, described here is an illumination/detection combination that exploits the speed of the TDI camera while enhancing the efficiency of the illumination. Line illumination is achieved by directly imaging the output of a two-dimensional light source, such as the output of a rectangular optical multi-mode fiber, to the sample plane at different magnifications for x and y. This solution comes with the numerous advantages.

The radiation source may have a rectangular shape, and multiple colors may be included in the radiation, either from a single multi wavelength source or multiple sources emitting different wavelengths.

The excitation system can effectively be used for multi-color imaging in a TDI-based scanning microscope for amplified single stranded DNA objects, e.g. amplified using rolling circle amplification, labeled with fluorescence nucleotides (FIG. 10) or for stained tissue sections (FIG. 11).

Accordingly, described here is an imaging system for imaging a biological sample or another sample containing fluorescent molecules. The system may include an optical system with a light source emitting light, wherein the light is directed by the optical system to the sample image plane, optionally via at least one plane being conjugate to the image plane. The optical source may be have an extended radiation pattern, in other words, the radiation beam may have an extent in the x-and y-planes, rather than a point source. The extent of the illumination region in x and y may be based on and matched to the detector area onto which the radiation may be imaged, preferably a TDI detector.

When the radiation is applied to the sample by the imaging system as described, it may illuminate a rectangular area which has a corresponding finite extent. The excitation radiation then causes fluorescence to be emitted from the biological sample. The sample has been previously tagged with at least one fluororphore, which fluoresces upon exposure to the excitation radiation. This fluorescence is then emitted back into the optical system, which images the fluorescent radiation onto the detector area of the TDI camera. The detector area is matched to the irradiation area on the sample, such that the camera area is effectively used.

In some embodiments, the system may also include at least one first detector that detects light from the sample transmitted through at least one mask, and at least one second detector that detects light from the sample reflected by the at least one mask. The system may also include a moving stage configured to continuously move the sample during detection by the first and second detectors, and an image processing controller, programmed to form a corrected image based on both the transmitted and the reflected light from the continuously moving sample.

Accordingly, described here is an optical imaging system having a focal plane and optical elements, for imaging a biological sample. The optical imaging system may include a stage holding the sample wherein the stage is configured to continuously move the sample in at least one direction parallel to the focal plane, at least one non-point-like light source emitting at least one wavelength. The system may further include at least one non-point-like light source disposed adjacent to a plane conjugate to the focal plane of the microscope, and a detector which forms an image of the sample on the moving sample stage. The detector may have at least one row of pixels to which the image of the sample moves perpendicular during the imaging process.

### BRIEF DESCRIPTION OF THE DRAWINGS

Various exemplary details are described with reference to the accompanying drawings which should not be taken to limit the invention to the specific embodiments shown but are for explanation and understanding only.

FIG. 1 is a simplified schematic illustration of a first exemplary optical layout 100 of the homogeneous line illumination using TDI-based continuous line scanning;

FIG. 2 is a simplified schematic illustration of a second exemplary optical layout 200 of the homogeneous line illumination exploiting spatial wavelengths separation;

FIG. 3 is a simplified schematic illustration of a third exemplary optical layout 300 of the homogeneous line illumination, using dichroic wavelengths separation;

FIG. 4 is a simplified schematic illustration of a fourth exemplary optical layout 400 of the homogeneous line illumination. FIG. 4A shows the optical diagram in a first dimension, and FIG. 4B shows the optical diagram in a second orthogonal dimension;

FIG. 5 is a simplified schematic illustration of a fifth exemplary optical layout 500 of the homogeneous line illumination. FIG. 5A shows the optical diagram in a first dimension, and FIG. 5B shows the optical diagram in a second orthogonal dimension;

FIG. 6 is a simplified schematic illustration of a sixth exemplary optical layout 550 of the homogeneous line illumination using TDI-based continuous line scanning, using diffraction grating for wavelength splitting;

FIG. 7 illustrates further detail of a prism-like wavelength splitting device;

FIG. 8 illustrates further detail of a mirror-like wavelength splitting device, using a mirror stack for wavelength splitting;

FIG. 9 illustrates further detail of a grating-like wavelength splitting device;

FIG. 10 shows an example of a homogeneous line illumination of two separated laser wavelengths (638 nm and 532 nm) at an intermediate image plane;

FIG. 11 shows the displacement of a plurality of homogeneously illuminated lines corresponding to 5 different excitation wavelengths for the corners of a rectangular optical fiber using the embodiment sketched in FIG. 1 (prism-based); and

FIG. 12 shows experimental data using the inventive technique for nucleotide imaging; and

FIG. 13 shows experimental data using the inventive technique for tissue imaging.

FIG. 14 illustrates the application of the novel imaging system to a generic downstream workflow;

FIG. 15 illustrates further detail of the downstream workflow genetic sequencing methodology coupled to the novel imaging system; and

FIG. 16 is an exemplary flow chart of the work process with the DNA sequencing strategy.

It should be understood that the drawings are not necessarily to scale, and that like numbers maybe may refer to like features.

### DETAILED DESCRIPTION

The following description is directed to a system for an optical sectioning microscope using TDI-based continuous line scanning, and its operation. In the embodiments described below, the following reference numbers may be used to refer to specific components. In the alternative embodiments, these components performing similar functions may use similar numbers. For example, in each embodiment, 10, 100, 200 and 300, the moving stage that moves the sample continuously may be designated 5.

The concept of the continuous line scanning optical sectioning with extended source microscope is first described generally. Thereafter, the figures illustrate several embodiments 100, 200, 300, 400 and 500 of this general concept. It should be understood that these embodiments are exemplary only, and that many variations may exist using these design principles.

| | |
|---|---|
| 5 | moving stage holding scanned sample |
| 8 | objective lens |
| 28 | wavelength splitting element |
| 30, 31, 32 | excitation radiation |
| 30′, 33, 34 | fluorescent radiation |
| 35 | emission filter changer |
| 36 | emission filter |
| 40 | radiative source |
| 42, 44 | cylindrical lenses |
| 52 | focusing lens |
| 54 | tube lens |
| 55 | intermediate image plane |
| 101, 102 | TDI camera |
| 70 | prism |
| 80 | wavelength dependent turning mirror |
| 90 | diffraction grating |
| 100-600 | exemplary embodiments |

In the discussion which follows, the term “conjugate plane” is used

synonymously with “image plane”, “intermediate plane” and “conjugate focal image plane” to refer to the plane of a non-magnified or magnified image of an object, such that the object and its image are interchangeable. An “intermediate image” refers to a pointwise image of a structure formed in the image plane by an optical system or an “intermediate image” refers to a pointwise image of a structure formed in a plane conjugate to the image plane by the optical system. A “tube lens” is a focusing element in a microscope positioned adjacent to the objective lens, which forms an intermediate image. The term “wavelength separating device” is used interchangeably with “wavelength splitting device” to refer to an optical element that applies different trajectories to different wavelength or frequency components of light. The term “anamorphotic” or “anamorphotically” refers to an optical element which changes the dimension of an image in at least one axis. “DAPI” is an acronym for (4′,6-diamidino-2-phenylindole) a blue-fluorescent DNA stain that exhibits ˜20-fold enhancement of fluorescence upon binding to AT regions of dsDNA. “Cy5” is a bright, far-red-fluorescent dye with excitation ideally suited for the 633 nm or 647 nm laser lines.

The techniques described here may be applicable to numerous sorts of biological samples and may be incorporated into a variety of workflows. For example, the samples may comprise a tissue section and may be at least about 1 μm thick. The sample and workflow may be configured for spatial proteomics, spatial transcriptomics and/or spatial genomics, or a combination thereof, for example. In these embodiments, the work flow may include staining of proteins using antibodies (proteomics) in tissue sections or single cells, for example, the staining of messenger RNA (transcriptomics) in tissue sections or single cells, for example and the staining of genetic material (genomics) inside the nuclei of a cell, for example. Additional details as to these workflows may be found in Patent Application serial numbers US 301,115 filed Nov. 13, 2018, EP21180189.9 filed Jun. 18, 2021 and EP 21198504.9 filed Sep. 23, 2021, each of which is incorporated by reference in their entireties for all purposes. Using the microscope described here would provide additional information as to the location of the stained structure. Sequencing of spatially located structure may also be performed by a cycling imaging approach of repeated staining and sequencing of the DNA or RNA. For example, to spatially locate 6 amino acids may require the six times repeated steps of staining, imaging, de-staining. Such cycling imaging is also possible to visualize multiple targets in spatial proteomics via staining, imaging, and de-staining.

Line illumination may be achieved by direct imaging the output of a rectangular optical multi-mode fiber to the sample plane at different magnifications for x and y. This solution comes with the following advantages:

Light efficient illumination. For a well-designed optical system, the excited area in the sample corresponds well to the imaged area by the detector without wasting light by exciting areas which are not detected. For common Gaussian illumination, it is not possible to restrict the illumination to the imaged part of the sample without blocking light.

Near homogeneous illumination. Homogencous illumination in x direction (i.e. perpendicular to the scan direction of the microscope) is desired for best quantifiability of the fluorescent image. Homogeneous illumination in y direction (i.e. the scan direction of the microscope) is desired to minimize non-linear effects like photobleaching, triplet-state saturation or sample damage (e.g. DNA-damage), which will all lead to a signal decrease and/or decrease of image quality.

The beam quality of the primary light source itself is not of concern since the illumination profile is determined by the fiber output only. Accordingly, cheap laser diodes can be used as light sources. They can be combined and coupled into one rectangular fiber of which the output is then imaged to the sample plane. In principle, any light source like solid state lasers, gas lasers, laser diodes, (multi-color) LEDs, halogen lamps, etc. can be coupled to the fiber and imaged to the sample plane.

Besides using an optical fiber output (as the preferred embodiment), the same beam shaping concept can be applied to any non-point-like (i.e. 2D extended) light source, e.g. a (multicolor) LED or laser diode.

For suppression of cross-talk between fluorescent channels and reduction of background, spectral separation via dispersion at a prism leads to side-by-side illumination at different wavelengths which can then be detected separately (as described in the patent applications MBG58 and particularly MBG59) via a line-type TDI camera.

Alternatively, the different wavelength can be split using a stack of one or more dichroics/dielectric mirrors at a slight angle which are placed conjugate to the pupil plane, or a diffraction grating, or any other wavelengths separating device.

While in principle a square fiber can be used, the use of a rectangular fiber of aspect ratio greater 1:1 reduces the demand on the optical system. For instance, the use of a rectangular fiber of aspect ratio 4:1 together with a magnification ratio of 8:1 in the optical system achieves an aspect ratio of the illuminated line of 32:1.

The use of an optical multi-mode fiber spatially decouples the primary light source(s) from the microscope because the light is guided through the fiber analog to electrical current through an electrical cable.

Laser illumination leads to speckles decreasing the homogeneity of the illumination. For mitigation of this usually undesired effect, it is possible to e.g. shake the fiber to scramble the speckles on short time scales, or use dynamic diffusers in the free-space beam path before and/or after the fiber, or use multiple, independent coherent sources for the same or similar wavelengths, or use sources with low coherence properties such as laser diodes, LEDs, or a combination thereof.

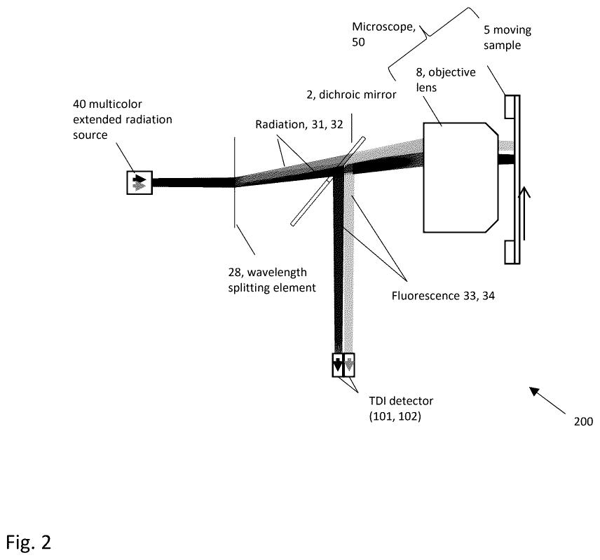

The output of the optical fiber is imaged at an aspect ratio of 1:8 (concept is not limited to this ratio and could work at almost any ratio) to the image plane of the microscope. The aspect ratio other than to 1:1 may be achieved using two nested telescopes of spherical or aspherical, but rotationally symmetric lenses (outer lenses) and cylindrical lenses (inner lenses) (FIGS. 2-4).

The system described here may generally include a biological sample mounted on a moving stage, the biological sample may have been previously stained with a fluorophore, which fluoresces upon excitation with the proper wavelength of light. The excitation light may be provided by an extended source of excitation radiation, wherein the radiation pattern falls over an extended area such as a rectangle with an extent in x-and y-. The system may further include a dichroic mirror that separates the outgoing excitation radiation with the incoming fluorescent radiation, and at least one TDT detector.

In the embodiments described below, the source of radiation may be a multicolor, extended radiation source. In other words, the radiation emitted from the source may have an extent in x and y (plane of the sample) and may include a plurality of wavelengths.

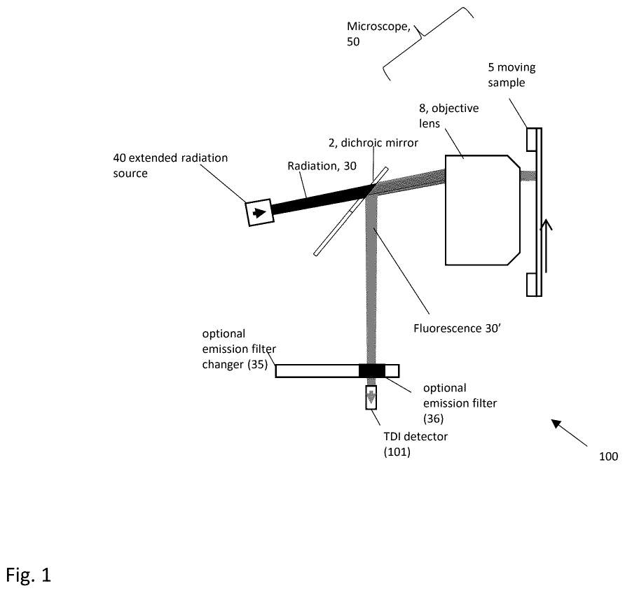

Various additional optical elements may be used to shape the beam profile and illuminated area, and these details are discussed with respect to the multiple embodiments shown in FIGS. 1-6.

The homogeneous extended illumination source may include first an optical excitation radiation source. The light source may provide input radiation or input light 30 to the optical microscope. The light may be provided using an optical fiber, such as a round or rectangular optical fiber 40. The light source launching the radiation into the fiber may be coherent such as a laser or incoherent, such as a LED. It should be understood that other radiation sources such as uncollimated light from an arc lamp, incandescent lamp or LEDs may also be used. The homogeneous illumination may alternatively be directly provided by single units or an array of LEDs or some incoherent illumination source, such as an incandescent lamp. The purpose of the excitation radiation is to excite one or more fluorescent tags that are affixed to the biological sample mounted on the movable sample stage, after transiting various beam shaping optical elements.

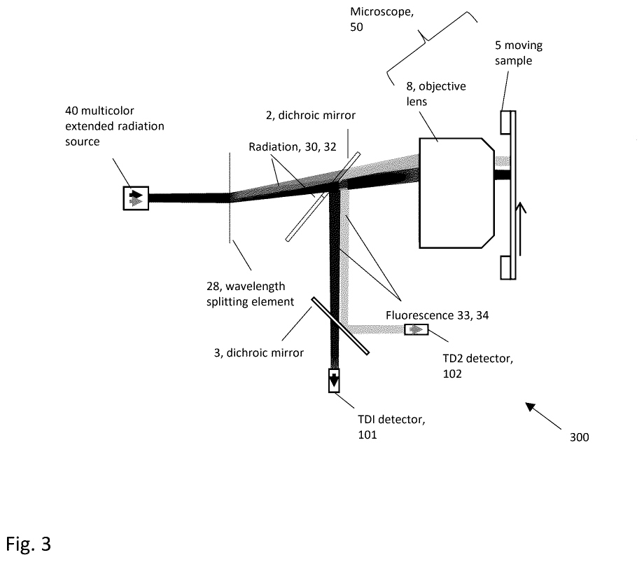

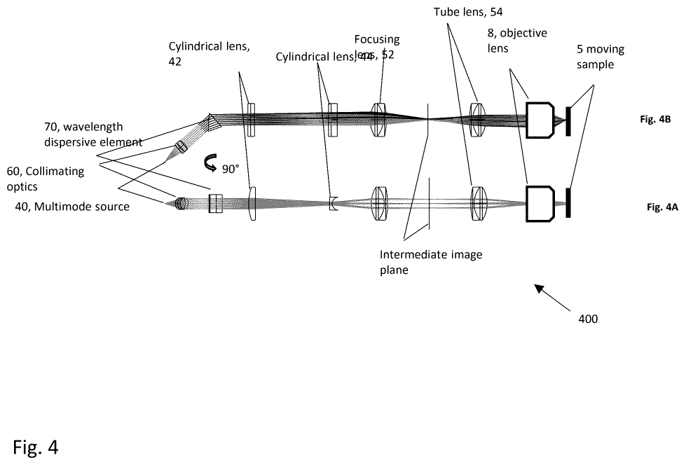

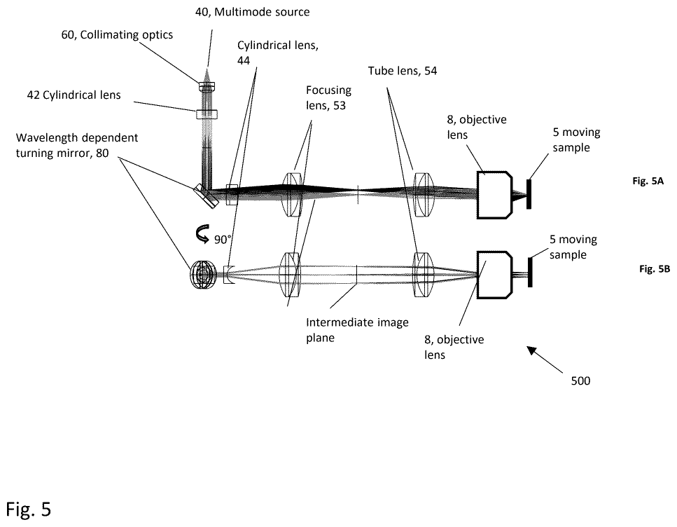

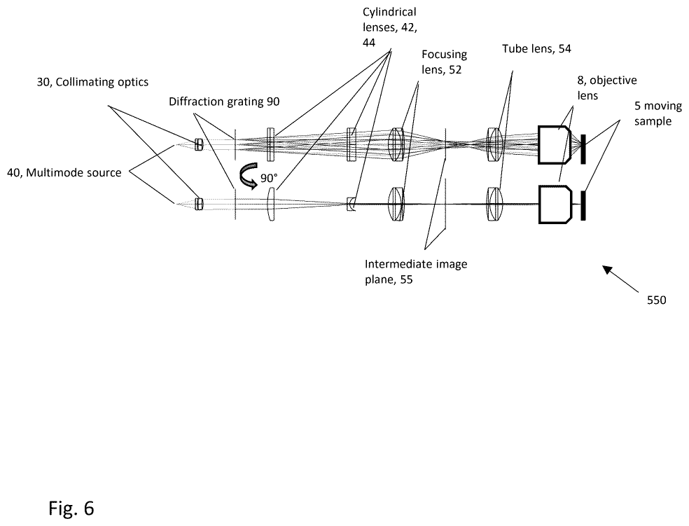

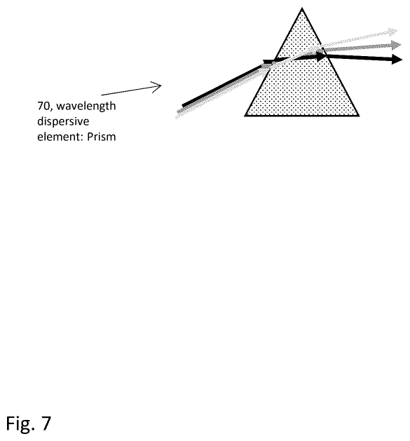

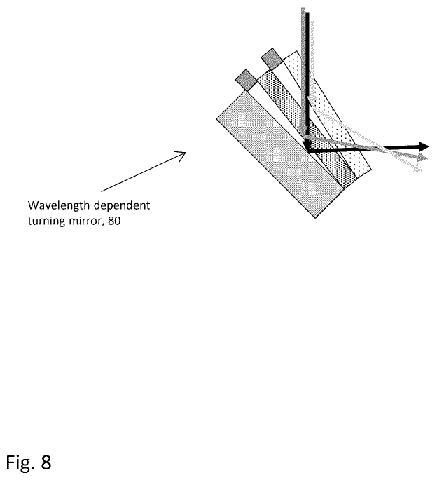

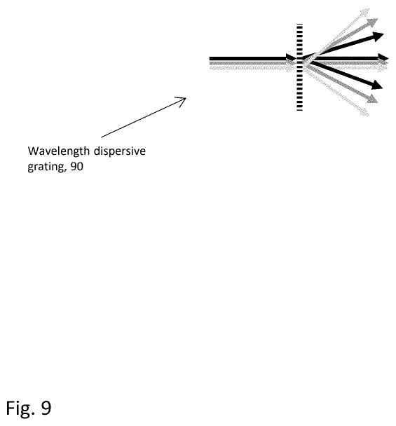

Upon exiting from the light source, the emitted light may diverge into a cone of light. In order to direct the radiation appropriately, a beam shaping optical structure may be provided, which may collimate or focus the diverging light and re-direct the radiation. In particular, the beam shaping structure may collimate and separate different wavelengths and re-direct the diverging light into generally parallel, divergent or convergent rays, which can be effectively focused, diverged or collimated by the downstream optical elements. In one embodiment, the beam shaping optics may include a pair of lenses and a wavelength separating component such as a prism, an amplitude grating, a phase grating or at least one dichroic mirror. The splitting of the wavelengths from the same light source (in this case the optical fiber) may either be achieved using a prism (FIGS. 2 and 5), a dichroic mirror stacked at a slight angle ona fully reflective mirror (FIGS. 3 and 6) or a diffraction grating (FIGS. 4 and 7), or another wavelength splitting device. In case of the prism, spatial separation of the illumination lines in the image plane (FIG. 8) can be achieved for typical wavelengths 405 nm, 488 nm, 532 nm, 561 nm, 640 nm, 785 nm all emitted by the same optical fiber or light source (FIG. 9). In case of the dichroic/mirror stack, a dichroic splits the wavelengths into two groups at each dichroic interface which will be separated spatially from each other in the image plane, if the stack is placed adjacent to a plane conjugate to the pupil plane.

The wavelength separating clement may be an element that refracts, transmits, diffracts or reflects light at an angle that depends upon the wavelength, such that relatively longer wavelength rays are diverted at a different angle than relatively shorter wavelength rays. These different trajectories may cause the wavelengths to propagate through the optical system differently, and as discussed in detail below, and in particular, the different wavelengths may impinge upon a different area of the sample. More generally, the wavelength separating device may be a component which directs different wavelengths of light into different directions or onto different positions in the image plane or a plane conjugate to the image plane.

As a result, the trajectories for different wavelengths of light may be different through the system, from upstream to downstream. The downstream components may be designed with attention given to the specific wavelengths of light which may interact with these components, and where these interactions may occur, in view of the different trajectories.

Continuing with the general description, upon emission by the source, and after collimation, diverging or focusing by the beam shaping optics, the radiation may impinge upon a dichroic mirror, which may have different optical properties for different wavelengths of light. In particular, the dichroic mirror may separate the excitation light from the different fluorescent wavelengths (compared to the excitation radiation), such that the different fluorescent wavelengths propagates along a different path than the excitation light, at the dichroic mirror.

This feature may be useful for separating incident excitation light from induced fluorescent light. From the backside, the excitation radiation may pass through the dichroic mirror,

As mentioned, this radiation may cause appropriately tagged molecules in the biological sample to fluoresce. Alternatively, embodiments that make use of phosphorescence may be possible. The fluorescence may then return along the reverse path as the excitation light. In particular, the fluorescence may travel back through the objective lens 8, back through the tube lens 12, and impinge upon the dichroic mirror, which may direct the fluorescent light into the TDI detector.

In some embodiments, the first imaging system and the second imaging system may be two TDI cameras, TDI camera 101 and TDI camera 102 or an appropriate setup of optical components 13 such as lenses that relay the image to two TDI cameras, TDI camera 101 and TDI camera 102. The TDI cameras 101 and 102 may be operated with a triggering that is chosen to be consistent with the movement of the sample stage, 5. In other words, the photoinduced charge in the photosensitive detector lines of the TDI camera 101, 102 is shifted synchronously with the sample movement such that light emitted by one point of the sample travels through the optical system at best photo-induces charge in the same, synchronously shifted, potential well that is being digitized after passing all photosensitive lines of the detector. Additional detail directed to these embodiments may be found in co-pending U.S. Provisional application Ser. No. 63/357,737, filed Jul. 1, 2022, and incorporated by reference in its entirety.

The discussion now turns to the detailed handling of the data as captured by the TDI camera. It should be understood that the one or more TDI cameras may all operate in essentially similar ways, such that the discussion to follow applies equally to the one or more TDI cameras. Importantly, the acquisition speed of both of these cameras is related to the velocity of the movement of the sample stage 5, which is holding the biological sample and moving it. A sample stage may be any structure capable of moving the sample predictably in the x-y plane (where z is the optical axis of the objective lens). Stepper motor driven stages, motorized gear-driven x-y stages, linear motor driven x-y stages, and other actuators holding the sample are examples of some workable movable sample stages.

This TDI detector 101, described elsewhere, accepts line by line intensity information from the moving sample 5. The TDI detector then puts out a series of images that are generated as the biological sample is scanned by sample stage 5. That is, rather than moving the excitation light over the biological sample on stage 5 to obtain a moving image, the stage 5 is moved through the rectangular excitation spot of the imaged excitation source 40.

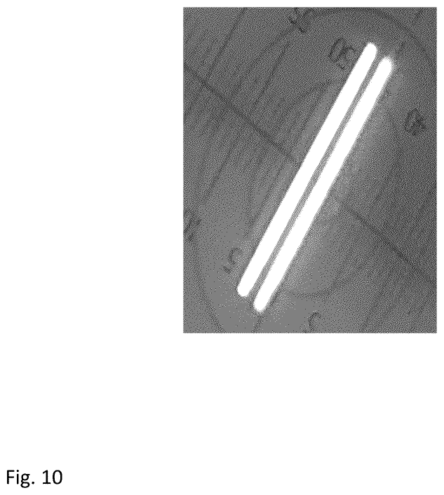

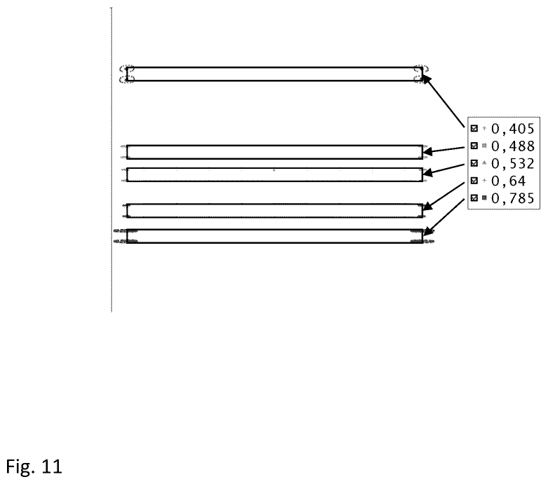

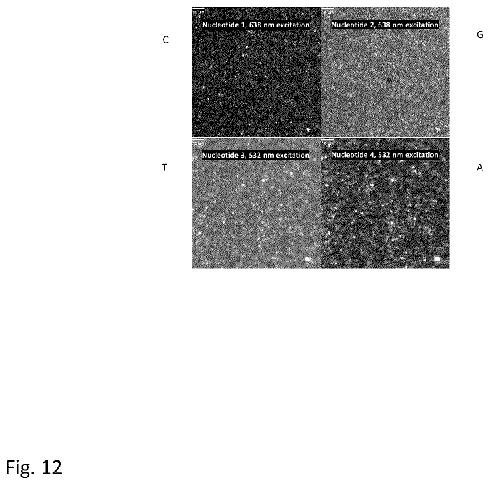

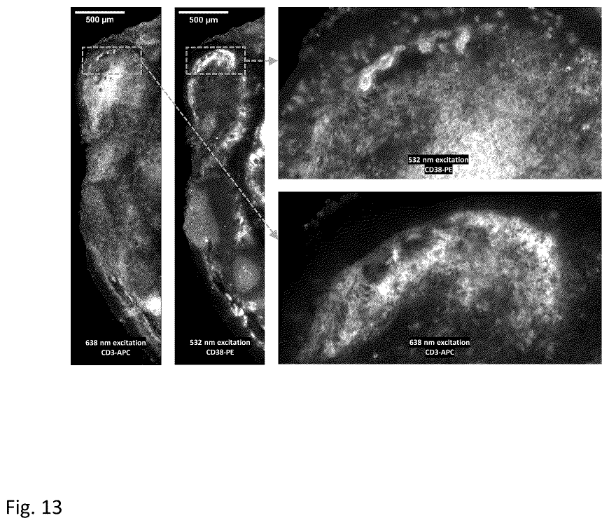

FIG. 1 is it simple first embodiment which uses a single wavelength to interrogate the biological sample. The next embodiment 200 shown in FIG. 2, uses a plurality of wavelengths to interrogate the biological sample embodiment, and a generic wavelength splitting device to separate the wavelengths. The next embodiment 300 shows an alternative optical arrangements for multiple detectors. The next embodiment 400 illustrated by FIG. 4 uses a prism as a wavelength splitting device. The next embodiment 500 illustrated in FIG. 5 uses a wavelength dependent turning mirror as a wavelength splitting device. The next embodiment 600 illustrated in FIG. 6 uses a diffraction grating as a wavelength splitting device. FIGS. 7-9 show additional details of the wavelength splitting options. FIGS. 10 and 11 illustrate the illumination patterns generated by the optical setups. FIGS. 12-13 shown experimental data-using the innovative systems.

Turning now to FIG. 1, FIG. 1 is a simple schematic illustration of the first embodiment 100 of a homogeneous line illumination system for multi channel scanning microscopy. FIG. 1 shows an extended radiation source 40, which may generate radiation 30 of a single wavelength such as a laser, or of multiple wavelengths. This radiation source 40 may have a finite extent in x-and y-as discussed previously, and may impinge through the backside of dichroic mirror 2, passing therethrough. The radiation 30 from source 40 then enters objective lens 80 of the imaging system 50. From objective lens 8, the radiation is directed onto a biological sample, which is mounted on a moving stage 5. Radiation from extended light source 40, is of a wavelength which may be absorbed by a fluorescent tag which is conjugated to the biological sample. The excitation radiation causes the tag to fluoresced at a different wavelength. This fluorescence 31 is emitted from the biological sample, and travels back through objective lens 8, and impinges on the front side of dichroic mirror 2.

Dichroic mirror 2 may be fabricated and oriented so as to reflect light or fluorescence emission from the sample through an optional emission filter (32) mounted in an optional emission filter changer (31) onto a TDI detector 101 shown at the bottom of the figure. This TDI detector 101, described elsewhere, accepts line by line intensity information from the moving sample 5. The TDI detector then puts out a series of images that are generated as the biological sample is scanned by sample stage 5. That is, rather than moving the excitation light over the biological sample on stage 5 to obtain a moving image, the stage 5 is moved through the rectangular excitation spot of the imaged excitation source 40.

The data generated by the moving image and the TDI camera may be enhanced using various techniques for noise reduction, elimination or suppression. These techniques may include a baseline subtraction or real time normalization, or pattern detection using artificial intelligence or machine learning or spectral manipulation such as frequency equalization or Fourier analysis. As mentioned previously, the frame rate of the detector may be based on the scanning speed of the moving stage, in order to generate a seamless image.

FIG. 2 shows the second embodiment 200 of the homogeneous line illumination for a multi channel scanning microscope. This embodiment uses multiple wavelengths of excitation light 31, 32 which are emitted by a radiation source 40. It should be understood that radiation source 40 may be multiple radiation sources, each emitting a different wavelength, or a single radiation source which is capable of embedding a plurality of wavelengths. In any case, a plurality of light wavelengths is emitted from the multicolor extended radiation source 40, and the source 40 has an extended emission profile when imaged on a plane.

The multicolor radiation 31, 32 then travels through a wavelength splitting element 28, which may split radiation 31 from radiation 32, and send them on different though perhaps adjacent, trajectories according to their wavelength. The wavelength splitting element 28 shown in FIG. 2 may use reflection, refraction, diffraction, phase delay, for example, or any other wavelength dependent discrimination means, to alter the trajectory of the radiation according to wavelength.

In the example shown in FIG. 2, two wavelengths of light are envisioned. However it should be understood that the concepts here can be extended to any number of different wavelengths, or continuously varying light source that emits a broad spectrum of wavelengths. In any event, the excitation radiation impinges on the backside of dichroic mirror 2, and travels unimpeded therethrough. Then, as in the first embodiment of FIG. 1, the radiation enters an objective lens 8, which focuses the light to the biological sample on the moving stage 5.

As before, the biological sample fluoresces as a result of the excitation radiation 31, 32 from multicolor extended radiation source 40. The biological sample fluoresces as a result of the excitation, and this fluorescent light 33, 34 is a different wavelength than the excitation light. This fluorescent light 33, 34 may travel back through objective lens 8, and may be reflected specularly from dichroic mirror 2. The specularly reflected light 33, 34 then travels to two TDI detectors 101, 102 which are placed adjacent to one another, in order to detect the fluorescent light 33, 34. The two wavelengths 33, 34 may be disposed adjacent to one another as shown in FIG. 2, and therefore may impinge on the TDI detector in an adjacent fashion as well. However, it should be understood that this is exemplary only, and the different wavelengths made be disposed in other positions relative to one another. For example they may cross one another and impinge on other regions of a TDI detector, or in different active regions of the same TDI detector.

FIG. 3 shows another embodiment 300 of the homogeneous line illumination for multichannel scanning microscopy system. This embodiment 300 is similar to the embodiment illustrated in FIG. 2, except for the detection scheme. This embodiment illustrates the various ways for detecting the fluorescence created by the multiple wavelength extended radiation source 40.

In FIG. 3, multicolor extended radiation source 40 again emits radiation of multiple wavelengths 31, 32, which traverses a wavelength splitting element 28. This wavelength splitting element 28 disperses the radiation along different trajectories for different wavelengths. In the example shown in FIG. 3, two wavelengths 31, 32 are launched on two different trajectories. Both of these wavelengths 31, 32 impinge on the backside of dichroic mirror 2, and pass unimpeded there through. As before, these two wavelengths enter objective lens 8, where they are collimated and focused onto the biological sample mounted on moving stage 5. To this point, this embodiment is similar to the embodiment shown in FIG. 2.

Upon being emitted from the biological sample, the fluorescent radiation 33, 34 passes back through objective lens 8, and impinges on the front side of dichroic mirror 2. The radiation may be specularly reflected into the TDI detector shown in FIG. 3.

Two TDI detectors are shown in FIG. 3, TDI detector 101 and TDI detector 102. In front of TDI detector 101 may be another dichroic mirror 3. This dichroic mirror 3 may pass radiation of the first wavelength 33, while reflecting radiation of the second wavelength 34. Accordingly, dichroic mirror 3 may allow fluorescent radiation 33 resulting from excitation light 31 to pass unimpeded through to TDI detector 101. Dichroic mirror 3 may then reflect fluorescent radiation 34 resulting from excitation radiation 32, to the second TDI detector 102. Accordingly, TDI detector 101 can be disposed at a right angle with respect to TDI detector 102. This may reduce the amount of cross talk between the two channels, being detected by the two separate detectors, TDI detector 101 and TDI detector 102.

FIG. 4 shows a fourth embodiment 400 of the homogeneous line illumination for multichannel scanning microscopy. In this embodiment, the wavelength splitting device 28 may be an optical prism which refracts light at different angles, as is known in the art. FIG. 4 shows two views of this embodiment, FIG. 4A and FIG. 4B. FIG. 4a shows the view of this embodiment in the first viewing plane, and FIG. 4B shows the same embodiment as viewed from an orthogonal viewing plane. For example, FIG. 4a may view the plane parallel to an optical bench, whereas FIG. 4b may view a plane perpendicular to the optical bench, i.e. the components as seen from above. Some features of the system may be easier to visualize in one of the two views, as will be clear from the discussion below.

As in the prior embodiments an extended radiation source 40 is provided, which may emit a plurality of radiative wavelengths. The source 40 may illuminate an extended, rectangular region, bathing the biological structures within that region in excitation light of different wavelengths. Each wavelength 31 may form its own rectangular illuminated region adjacent to the other 32 in the far field, as was shown in FIG. 2.

For simplicity, as FIGS. 4 and 6 do not show the different trajectories of the different wavelengths downstream of the wavelength separating structure 28, but FIG. 5 does show the different trajectories for two wavelengths. Since FIGS. 4 and 6 depict ray traces through the system, the figures would become exceptionally complex, if the different trajectories of the different wavelengths were also shown. But it should be understood that the wavelength splitting element 28 operates in these embodiments largely as they did before and shown in FIGS. 2, 3 and 5.

As before, radiation is emitted from the extended source 40 and traverses collimating optics 60 before impinging on the wavelength dispersive element, here an optical prism 70. The prism 70 is an embodiment of the wavelength dispersing optical element, but it should be understood that other wavelength dispersive optical elements could also be used in its place. The optical prism 70 refracts light of different wavelengths according to Snell's law, sending the radiative components along different trajectories as a result of refraction at the surface of the prism. Accordingly, the wavelengths are separated in space upon passing through the prism 70.

The light that impinges on set of cylindrical lenses 42 and 44 as shown. The two cylindrical lenses may be arranged as a telescope such that they change the magnification in one spatial direction, but not in the perpendicular spatial direction. In other words, they may change the diameter of the excitation light beam collimated by the collimating optics 60 in one spatial direction and leave the diameter of the collimated light beam unaltered in the other spatial direction while maintaining collimation after the passage through both cylindrical lenses. Accordingly, the image of the spatially extended light source formed by the focusing lens 52 in the intermediate image plane may have a different magnification for two orthogonal spatial directions.

FIG. 4B shows the same optical arrangement us FIG. 4A but as viewed in the orthogonal plane. Accordingly, FIG. 4B is the view of the optical set up as seen from above. As before in FIG. 4A, the multimode source 40 emits light which then passes through the wavelength splitting element, here a prism 70. After passing through the prism 70. the light passes through the first of two cylindrical lenses 42, 44. Upon exiting the tube lens 54, the light is again directed into the objective lens 8, and then focuses it on the biological sample supported on the moving stage 5.

FIG. 5 shows a fifth exemplary embodiment 500 of the homogeneous line illumination system, as applied to multi channel scanning microscopy. In this embodiment, a wavelength dependent turning mirror is used to separate the wavelengths from the multicolor radiation source 40.

As before, FIG. 5A shows the optical layout in a first viewing dimension, and FIG. 5B shows the same optical layout in a second orthogonal viewing dimension. FIG. 5A shows the optical system from above, that is, in a plane orthogonal to the reference plane (e.g. optical table) holding the optical elements.

Turning now to FIG. 5A, the multi mode source 40 is depicted emitting radiation of multiple wavelengths into collimating optics 60. Different trajectories for two different wavelengths are shown in FIG. 5A and 5B, forming spatially separated images at the intemediate image plane, since the two different wavelengths are focused above each other in 5B.

Collimating optics 60 may collimate the radiation and apply it to a first cylindrical lens 42. The first cylindrical lens 42 may focus or diverge the light in one dimension but not the other. The multiple wavelengths then impinge upon the wavelength dependent turning mirror 80. Turning mirror 80 may redirect the radiation at an angle that depends on the wavelength of the radiation. This wavelength dependent turning mirror 80 is shown in more detail in a later FIG. 6.

From turning mirror 80, the radiation may travels through a second cylindrical lens 44, which may collimate the beam and apply it to focusing lens 52. Focusing lens 52 may then create an intermediate image plane 120 for the light, before sending it for re-imaging by the tube lens 54 and the objective lens 8 onto biological sample on moving stage 5. Objective lens 8 then focuses the radiation onto the biological sample which is supported by the moving sample stage 5.

Note that in contrast to using infinity corrected objective lenses as shown in FIGS. 4, 5, 6, which form images at a finite distance when operated together with at least one tube lens 54, it is also possible to omit the use of a tube lens and use objective lenses with certain focal planes at both sides as shown in FIGS. 1, 2, 3.

FIG. 5B shows the same components as seen from the orthogonal view and dimension. In other words, FIG. 5b shows the optical system in a plane perpendicular to the supporting table holding the optical elements. The ray tracing diagram of FIG. 5B is similar to that shown in FIG. 5A, with the radiation emitted by the multicolor source 40 impinging on the collimating optics 60 and cylindrical lens 42 and turning mirror 80 are essentially collocated in this view. Accordingly, the multi mode source 40 the wavelength dependent turning mirror 80, the cylindrical lens 42, and the wavelength dependent turning mirror 80 all appear as a single structure in FIG. 5B.

From the wavelength dependent turning mirror 80, the radiation passes through the second cylindrical lens 44, which collimate the beam and apply it to focusing lens 52. Focussig lens 52 may then create an intermediate image plane 120 for the light, before sending it for re-imaging by the tube lens 54 and objective lens 8 onto biological sample on moving stage 5. Objective lens 8 then focuses the radiation onto the biological sample which is supported by the moving sample stage 5.

FIG. 6 shows another exemplary embodiment 550 using yet another example of a wavelength separating element. In this embodiment, the wavelength separating element is a diffraction grating 70, as is known in the art. Diffraction grating 70 diverts the radiation into a trajectory which depends on its wavelength. Accordingly, diffraction grating 70 separates the different components of the multicolor radiation source 40. In other ways this embodiment is similar to other shown FIGS. 1-5. The diffraction grating may e.g. be a one-or two-dimensional an amplitude modulation element such as a grid, a one-or two-dimensional phase modulation element such as a grid, a volume holographic clement or of other type.

From the multi mode radiation source 40, multiple colors of radiation are sent to collimating optics first. The divergent light source 40 is there by converted into a collimated beam. The collimated beam then impinges on diffraction grading 70 which separates the wavelengths as discussed above. Each wavelength, now on its own trajectory, follows through the rest of the system according to ray tracing optics, as is known in the art. From the diffraction grating 70 the radiation impinges on a first cylindrical lens 42 which focus or diverges the radiation in one dimension but does not alter it in the other dimension. The second cylindrical lens collimates the radiation which then passes through focusing lens 52. The light is then focused on the intermediate image plane as shown and relayed to the sample at a certain magnification by the tube lens 54 and the objective lens 8, while the sample is supported on the moving sample stage 5.

Sample stage 5 can be any sort of stage which is movable in the x-y plane. This could be a stepper motor for example or a rotary actuator or any other sort of linear or rotary actuator as it known in the art. Importantly, the velocity of the moving stage, is related to the triggering of the TDI camera that is used for the detection as discussed previously.

For clarity, in the aforementioned embodiments, 400-600, the TDI detectors are not shown explicitly. It should be understood that embodiments 400-600 are intended to use at least one TDI camera 101, and possibly any number of additional cameras. It should be understood that although the detection systems the TDI detector 101 and 02 are not shown in FIGS. 4-6, they should nonetheless be understood to be there as described above.

Accordingly, one or more TDI cameras may be used to image the fluorescent radiation resulting from the excitation of the biological sample. This radiation may be a result of fluorescent dye affixed to the molecule which radiates as a specific known wavelength upon excitation by the excitation source. This wavelength, of course, is a different wavelength than the excitation wavelength and thus is treated differently by the optical components in the optical path.

FIG. 7 shows in detail the function of the wavelength dispersive element 28, in the case where the wavelength dispersive element is an optical prism 70. As known in the art, an optical prism has slope slanted surfaces which refract radiation according to Snells law, and thus according to the wavelength of that radiation. The two surfaces are the entrance surface and the exit surface, both working together to separate the different wavelengths in a different trajectories as they exit the prism. This behavior results in the well-known phenomenon of prismatic dispersion of multicolored light.

FIG. 8 shows in detail the function of the wavelength dispersive element 28, in the case where the wavelength separating element 28 is a wavelength dependent turning mirror 80. FIG. 8 shows greater detail of the working principle behind the wavelength sensitive turning mirror 80 shown in FIG. 5. The construction of such a wavelength dependent turning mirror is shown in this FIG. 8, however these details should be understood to be exemplary only. Any other sort of wavelength separating device may be used in the place. As shown in this figure, multiple reflective films may be positioned one substantially adjacent to the other but separated by a wedge of either transparent material or air. Accordingly, each layer in this mirror has a variable distance from the reflective surface of its adjacent layer. Because of this angular deviation, the light that is specularly reflected off the surfaces of this film may be directed into a different trajectory than other wavelengths. Once again it should be understood that these details are exemplary only.

FIG. 9 shows greater detail of a third example of a wavelength dependent separating optical clement 28. In this embodiment. the wavelength separating optical element 28 is a diffraction grating 90. Diffraction grating 90, or a series of opaque and angled reflective regions, disposed adjacent to one another, such that they reflect or diffract light as it passes through. Diffraction gratings result in a series of maxima and minima in the far field of the diffraction grating that correspond to areas of destructive and constructive interference from the optical element. Diffraction gratings are well-known in the art, and this will not be described further here. Further details can be found in numerous sources for example in Wikipedia (https://en.wikipedia.org/wiki/Diffraction_grating).

FIG. 10 shows an optical image of the homogeneous line illumination which is generated by the radiation source 40 as described here. This figure is a photograph of the radiation as it impinges upon a screen in the far field disposed at the intermediate image plane of the embodiment depicted in FIG. 4. As shown in the photo, the radiation has a large aspect ratio of at least 10:1 to one and more preferably about 32:1, resulting in a long homogeneous rectangular area of illumination that is applied to the sample. Importantly, the aspect ratio of this illumination area is related to the aspect ratio of the detection area of the TDI cameras used to detect the fluorescent pattern caused by the excitation light. In other words, the line radiation of the source as imaged on the sample is matched to the fluorescent radiation as it impinges on the detector in terms of the aspect ratio of that radiation. Further details of the detection system can be found in co-pending U.S. patent application Ser. No. 63/357,737. This United States patent application is incorporated by reference and its entirety for all purposes.

FIG. 10 shows two similar homogeneous rectangular illumination patterns one adjacent to the other. The first illumination pattern corresponds to a first wavelength from the multi color wavelength source, whereas the adjacent line pattern illumination corresponds to the second wavelength 32 in the multicolor radiation source. These two adjacent rectangular areas are the result of separation by the optically dispersive wavelength dependent element 28 which is disposed upstream the system.

FIG. 11 is similar to FIG. 10, however it is illustrates the concept of schematically rather than photographically, as modelled by a ray tracing program. FIG. 11 shows numerous homogeneous line illumination patterns generated by a plurality of wavelengths from the multicolor extended radiation source 40. The first wavelength is 405 nm and that radiation is focused again into a rectangle which is shown at the top of the figure. The other rectangular illumination patterns correspond to the other four wavelengths in the multicolor radiation source, 488 nm, 532 nm, 640 nm and 785 nm. Each of these wavelengths travels through the same optical systems but falls on adjacent spots, because of the dispersion at an optical element located upstream.

FIG. 12 shows actual data from this homogeneous line scanning multichannel microscope system which has been described previously. FIG. 12 shows four quadrants of data each showing the fluorescent pattern resulting from excitation by two different wavelengths as described previously which have been further split due to their emission characteristics at different fluorescence wavelengths in the detection optics. As clear from the photo, different structures show up differently because of their conjugation to different fluorescent tags.

Each of the quadrants of FIG. 12 corresponds to an image of the fluorescent labels of a different oligonucleotide, thymine (T) adenine (A) guanine (G) and cytocine (C). Accordingly, the techniques described here allow the formation of images showing the locations of specific amino acids and thus are directly applicable to genomic sequencing or spatially resolved transcriptomic sequencing after encoding RNA into DNA information.

FIG. 13 shows actual data from this homogeneous line scanning multichannel microscope system which has been described previously.

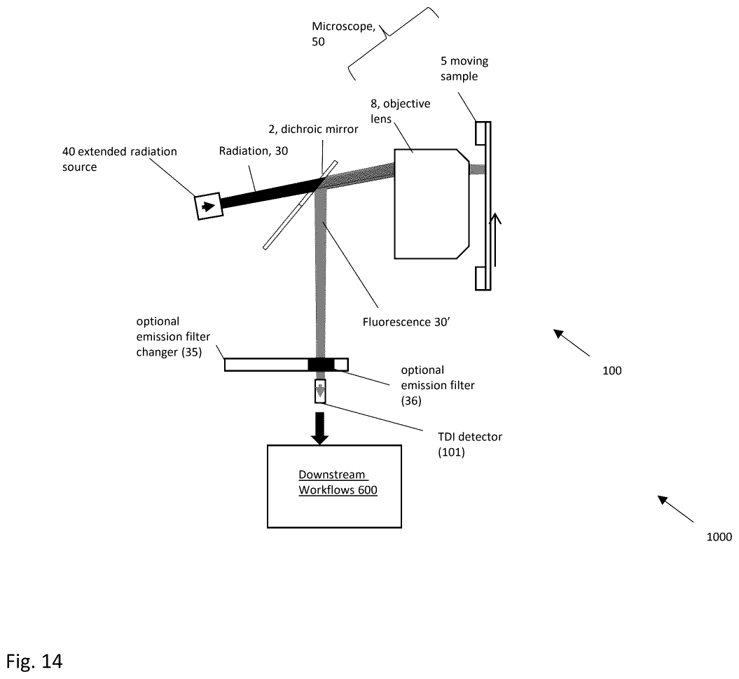

FIGS. 14-16 illustrate the application of this imaging system 100 to a generic downstream workflow 600. Many different sorts of downstream workflows are envisioned, including centrifugation, incubation, staining, chemical and microscopic analysis. One embodiment is discussed in detail below, which is a DNA analysis protocol.

The microscope can be used for imaging fluorescently labeled samples (5) such as adherent cells, viruses, bacteria, co-cultures, tissue sections, whole organs, or organoids.

To image multiple different targets in a sample, e.g. biomolecules such as proteins, nucleic acids, lipids, cellular organelles, receptors, such targets may be stained with different fluorophores.

Samples (5) may be chemically fixed before being put on the microscope or being put live on the microscope (50).

For imaging of live samples, the sample may be embedded in a device to keep the sample alive such as an incubator which includes the whole microscope or which is directly attached to the microscope stage.

Adherent cells, such as immortalized cell lines, may be grown at 0 to 100% confluency on coverslips, slides, petri dishes or other biocompatible vessels.

Co-cultures can be at least two types of different adherent cells, viruses, bacteria or other biological targets or a combination thereof simultaneously being imaged.

Imaging of co-cultures is typically performed to investigate the interaction between at least two different biological targets.

Tissue sections may be samples taken from human or animal organs.

Possible preparation techniques for tissue sections include cryo sectioning or paraffin embedding.

Whole organs may be taken from humans or animals and prepared e.g. using established fixation and/or optical clearing techniques before being fluorescently labeled and applied to the microscope.

Organoids may be artificially prepared, multicellular microstructures e.g. to mimic physiologically relevant human or animal organs.

Fast stage scanning of the whole sample is beneficial for achieving high throughput or scanning the whole sample or parts of the sample multiple times at high frequency to achieve live imaging at high temporal resolution.

High throughput may be desired when multiple targets such as proteins, nucleid acids, lipids, organelles, receptors, or any combination thereof in the same sample shall be visualized, e.g. in a cycling imaging approach.

A possible implementation of a cyclic imaging approach includes in each of multiple cycles the targeted staining of a subset of the multiple targets e.g. including immunostaining followed by their imaging followed by the erase of the staining (i.e. de-staining) in the particular cycle.

Multiple targets may be visualized simultaneously, e.g. using any of the spatial wavelengths separation approaches shown in FIG. 2), (3), (4), (5) or (6) and/or subsequently inserting an emission filter changer (35) before the detector (101).

An emission filter changer may be a wheel, a slider, or any other device that holds at least one emission filter to spectrally filter the light emitted from the sample which passes to the detector. Such at least one emission filter can actively be moved into the optical path by the emission filter changer.

When using an emission filter changer (35) before the detector (101), the one filter or multiple emission filters (36) can be selected according to the emission wavelength of the target fluorophore.

An alternative approach to visualize multiple targets simultaneously is to use a light source (40) that can emit different wavelengths and at least two different wavelengths can be individually switched on or off. The sample can be scanned multiple times, each time switching one or a different combination of individual wavelengths on and exciting different fluorescently labeled targets in the sample by spectral targeting.

Spectral targeting is possible as a set of different fluorophores for staining the sample can be selected carefully according to their excitation spectrum. Hence, each target can be excited at a different peak wavelength.

Efficient targeting is possible by a combination of simultaneous and subsequent imaging of different fluorophores and/or targeting the emission by selecting the corresponding one emission filter or emission filters, respectively, and/or targeting the excitation by selecting the corresponding excitation wavelengths or a combination thereof.

The tissue samples imaged by imaging device 10, described above may be coupled to a genetic sequencer, simply referred to as sequencer 600, or other cellular or genetic manipulation, and thereby obtain detailed information relating to a singular, specific biological particle, cell, tissue section, whole organ sample, organoid or any of the above mentioned.

The system 1000 is shown in FIG. 14, with the sequencer 600 coupled to the Imaging device 10, to create the Imaging device and sequencer 1000. It should be understood that although the system is described with respect to imaging system 10, the concepts disclosed here may also be applied to imaging systems 100, 200 and 300, as well as other sorts of imaging systems. In addition, an identifying label or barcode may be affixed to the particle, such that the genomic sequence is associated with a single, identified, particular biological particle.

This whole single tissue sample sequencer 1000 may operate generally as follows:
1) Put a sample and chemistry including the barcode information into a sample receptacle
2) Image the sample
2) Lyse sample to set DNA and RNA free
3) Fragment the DNA/RNA, optionally followed by some further chemistry. Label fragments of DNA/RNA with barcode information
4) Sequence fragments and via barcode to find out what specific tissue sample had what genetic information. Sequencing may make use of a genetic library, depending on the sequencing technique. These steps and techniques are described in further detail with respect to the embodiment discussed below. The details of these steps can also be found in the following documents, all of which are incorporated by reference in their entireties.
1) “Methods and Systems for Associating Physical and Genetic Properties of Biological Particles” PCT/US2018/061629, 16 Nov. 2018 (WO2020207963)
2) “Conjugates Having An Enzymatically Releasable Detection Moiety And a Barcode Moiety” (PCT/EP2020/059747, filed Apr. 6, 2020 (WO 2019099908)
3) “COLOR AND BARDCODED BEADS FOR SINGLE CELL INDEXING” 12 Nov. 2020, PCT/EP2020/081851
4) EP20182775.5 “METHOD COMBINING SINGLE CELL GENE EXPRESSION MAPPING AND TARGETED RNA OR c-DNA SEQUENCING USING PADLOCK OLIGONUCLEOTIDES COMPRISING A BARCODE REGION” June 29 2020, EP20182775.5

What follows is an embodiment of the system and method outlined generally above. In the following description, certain terms of art may be used. While these terms are widely known to those skilled in the art, to avoid confusion the following definitions are offered:

Genomics: A part of molecular biology looking at the structure, function, evolution and mapping of genomes of any living organism. It includes the study of a set of DNA and/or genes.

Transcriptomics: The study of a set of RNA molecules in cells and/or tissue.

Proteomics: Studying the set of proteins in cells and/or tissue.

cDNA is complementary DNA, which is DNA synthesized from a single-stranded RNA (e.g., messenger RNA (mRNA) or microRNA (miRNA)) template in a reaction catalyzed by the enzyme reverse transcriptase.

Barcoded primers are single ended oligonucleotides that contain predefined sequences. These sequences can later on be decoded again and can be used as a unique identifier for each detected tissue sample in the process. In the present application barcoded primers contain oligo (dT) which will interact with the poly A tail of the mRNA, a unique barcode and molecular identifier (UMI).

Reverse transcription (RT) reagents are all reagents used to do reverse transcription of RNA to cDNA. Usually RT reagents contain an enzyme such as reverse transcriptase, random hexamers, oligo (dT) and sequence specific reverse primers.

Reaction vesicles are the reactors where the reaction takes place. In this application reaction vesicles are the water in oil tissue sample with the bead and tissue sample.

NGS stands for next generation sequencing and allows the determination of sequences in a massively parallel manner.

RCA stands for rolling circle amplification. It is a method of isothermal amplification of circular DNA molecules.

Rolonies are the product of the RCA process.

Poly A-tailed means the polyadenylation of a RNA transcript. Poly A-tail sequences only contain adenine bases.

Adaptor oligos are used during library preparation for sequencing. Adapter oligos allow to fish out short target DNA sequences of interest.

SPRI beads stands for solid phase reversible immobilization beads. Those beads may be magnetic with a carboxyl group coating and are able to bind DNA. SPRI beads can therefore be used to do size selection.

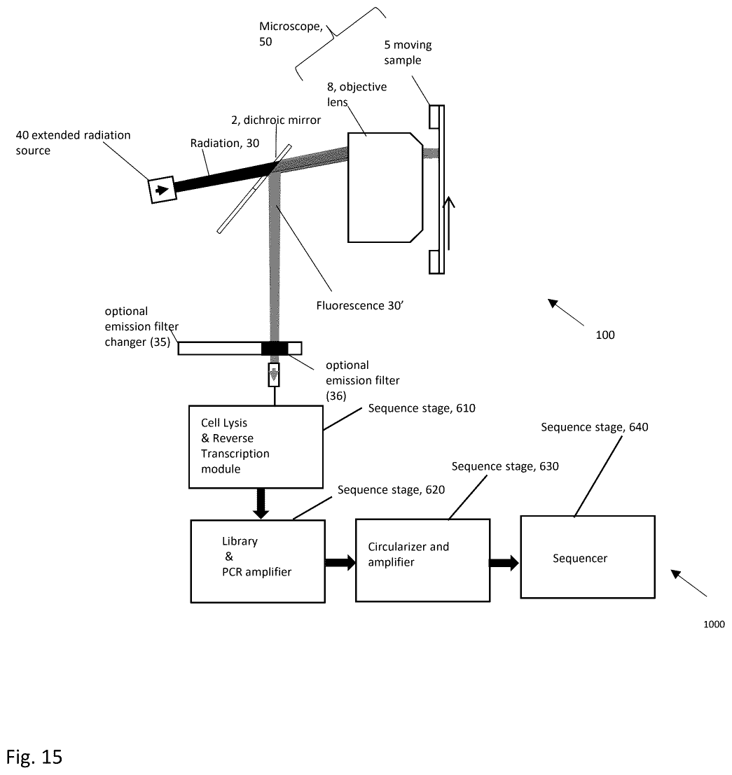

In FIG. 15, the sequencer is further depicted as including a number of modules 610-640. It should be understood that not all of these modules may be necessary to practice this invention, but that FIG. 15 is merely illustrative of a sequencing embodiment. First, each sample may further encapsulate a barcoded bead 610.

Each bead encapsulated in the samples contains many barcoded primers. The beads provides primers that contain oligo (dT) which will interact with the poly A tail of the mRNA, a unique barcode and molecular identifier (UMI) that are used to index the 3′ end of cDNA molecules during reverse transcription, thus enabling the assignment of every individual transcripts and individual samples and finally the primers provide by the beads contain a PCR handle for further amplification of the library construct.

The sequencer 600 may further include a sample lysis & RT stage 610. Each functional sample may contain a single cell, a single bead with primers as described in 610, and RT reagents. Within each reaction vesicle, a single sample is lysed and reverse transcription of polyadenylated mRNA may occur. As a result, all cDNAs from a single sample will have the same barcode, allowing the sequencing reads to be mapped back to their original single samples of origin. After that step the samples are pooled together and a alcohol based reagent is added to dissolve the oil water tissue sample solution. A washing step is introduced to get rid of unwanted leftovers. The preparation of NGS libraries from these barcoded cDNAs is then carried out in a highly efficient bulk reaction.

The sequencer 600 may further include a library preparation stage, 620: The barcoded double stranded cDNA are used to prepare an NGS library using conventional and prior art approach. The cDNA is fragmented enzymatically and post fragmentation, the ends are repaired and poly A-tailed. Adaptor oligos are then ligated to each extremity clean up with SPRI beads and amplified by PCR.

The sequencer 600 may further include a circularization and amplification, stage 630: The cDNA library containing adaptors is then used as a template for rolling circle amplification (RCA). The RCA reaction needs to be primed using an oligonucleotide (RCA primers) that is complementary to the common adapter portion of the circularized DNA library. This short duplex/circular template is recognized by the Polymerase performing the RCA which amplifies the DNA regardless of the target sequence into DNA rolonies containing several hundred copies or concatemers of the DNA.

The sequencer 600 may further include a Sequencing stage, 640: The rolonies are then loaded in to a micro fluidics channel. The rolonies will randomly immobilize on a functionalized glass surface. Multiple different chemistry reagents are sequentially applied to sequence the bases on each rolony. The bases are labeled with fluorescence dyes which an optical imaging system can detect during each cycle of sequencing. A sophisticated algorithm takes all those raw images coming from the optical imaging system and does the base calling for each rolony and determines the bases.

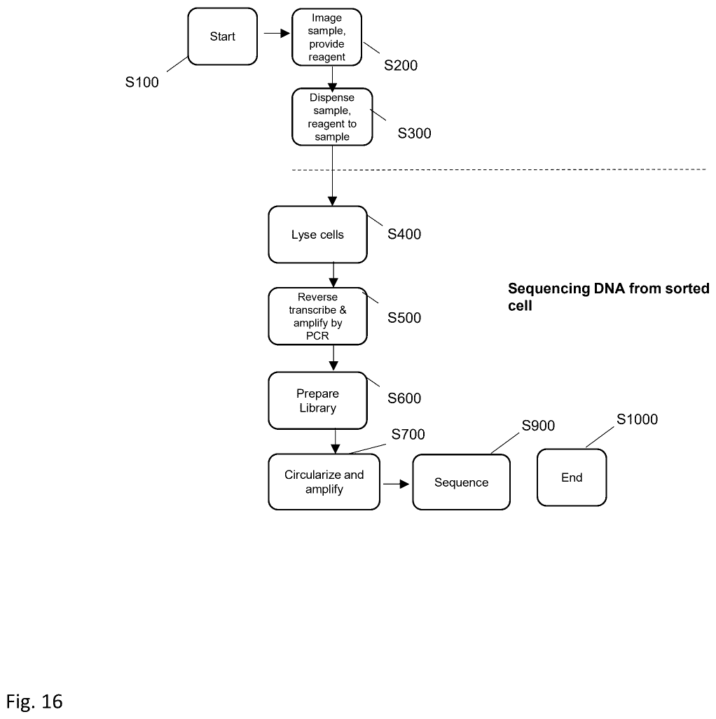

A process or method to sequence the genetic material of a single biological particle separated from the sample after imaging by the imaging device 10 is also disclosed here, and this method is illustrated in FIG. 16. The method may begin in step S100. In step S200 the samples is imaged. In step S300, the sample is dispensed. and inserted into a flowing stream of an immiscible fluidIn step S400, the tissue sample is destroyed, and the tissue sample is lysed to release the genetic material encapsulated therein. In step S500, the genetic material is reverse transcribed and amplified by polymerase chain reaction. In step S600 the cDNA libaray is prepared. In step S700, the genetic material is circularized and amplified in an RCA. In step S900, the sequence is ascertained by successive application of a fluorescent reagent, and imaging of the sample.

It should be understand that not all of these step need necessarily be performed, and they may not need to be performed in the precise order given in FIG. 16. Furthermore, each of these steps may include a number of sub-steps. For example, in step S900 “sequence”, the sample of genetic material may first be introduced into a microchannel and then immobilized on a functionalized glass surface.

Accordingly, disclosed here is a novel imaging system that enables enhanced throughput and an extended radiation source, and this system is applied to a DNA sequencing methodology. The sample dispenser may include a microfluidic channel formed in a substrate, a first fluid, including at least one target particle and at least one bead and non-target material, a microfabricated MEMS fluidic valve, configured to open and close the microfluidic channel and formed in the same substrate wherein the MEMS valve when in the sort position, separates the target particle and redirects the target particle into a first sort channel containing the first fluid, a second fluid, immiscible with the first fluid. The second fluid may be contained in a second microfluidic channel containing the second immiscible fluid, a nozzle disposed between the first sort channel and the second microfluidic channel, wherein the nozzle forms a tissue sample comprising a quantity of the first fluid along with the target particle, the quantity determined by the MEMS fluidic valve opening and closing, a fluidic manifold that accepts the tissue sample and lyses the target particle enclosed within the particle to release genomic material; and a sequencer that sequences the genomic material.

The imaging system can be used in a multiomics application combining workflows of genomics and transcriptomics, genomics and proteomics, transcriptomics and proteomics, or transcriptomics, proteomics and genomics. When applied to tissue samples, organs, or organoids, biological information derived from the images can be put into spatial context and analyzed therein.

Accordingly, disclosed here is an optical imaging system having a focal plane and optical elements, for imaging a biological sample. The systema may include a stage holding the sample wherein the stage is configured to continuously move the sample in at least one direction parallel to the focal plane, and at least one non-point-like light source emitting at least one wavelength. The system may further include at least one non-point-like light source disposed adjacent to a plane conjugate to the focal plane of the microscope, and a detector which forms an image of the sample on the moving sample stage.

The detector may have at least one row of pixels to which the image of the sample moves perpendicular during the imaging process. The detector may be a time delayed interval (TDI) detector.

The at least one non-point-like light source may emit a plurality of wavelengths, and the non-point-like light source may be at least one of a laser, an array of lasers, an optical fiber, an array of optical fibers, a laser diode, an array of laser diodes, an LED, an array of LEDs, an incandescent lamp, an array of incandescent lamps, a gas-discharge lamp, and an array of gas-discharge lamps. The at least one non-point-like light source may be anamorphotically imaged onto a sample plane containing the biological sample.

The imaging system may further comprise an optical imaging system having a focal plane and optical elements, for imaging a biological sample, comprising: a stage holding the sample wherein the stage is configured to continuously move the sample in at least one direction parallel to the focal plane; at least one non-point-like light source emitting at least one wavelength; at least one non-point-like light source disposed adjacent to a plane conjugate to the focal plane of the microscope, and a detector which forms an image of the sample on the moving sample stage, wherein the detector has at least one row of pixels to which the image of the sample moves perpendicular during the imaging process. This imaging system may then be coupled to a downstream workflow, including for example, and DNA sequencing system. The workflow may include flow may include antigen retrieval of tissue, tissue lysing, padlock probe hybridization and ligation on RNA or reverse transcription of RNA, and circularization of the padlock probe and enzymatic amplification.

The system may further include a wavelength separating element. he wavelength separating element may separates wavelengths by diffraction, refraction, transmission or reflection. The wavelength separating element may be at least one of a prism, a wavelength dependent turning mirror and a diffraction grating. The wavelength separating structure may include an adjacent angled mirror with a plurality of surfaces, wherein at least one surface is dichroic. The optical elements may include at least one optical element chosen from the group consisting of a light amplitude modulating element, a light phase modulating element, a light refracting element, and wherein at least one optical element is disposed in an intermediate image plane of the optical imaging system. In some embodiments, beam shaping optics may include a pair of lenses and a wavelength separating component such as a prism, an amplitude grating, a phase grating or at least one dichroic mirror. The intermediate image plane may also in image plane of the detector.

A DNA sequencing system may use the optical imaging system described here. A RNA sequencing system may use the optical imaging system described here. A system for performing spatial transcriptomics may use the optical imaging system described here. A system for performing spatial genomics, may use the optical imaging system described here.

A system for performing spatial proteomics may use the optical imaging system described here. A system for imaging of cell monolayer, may use the optical imaging system described here. A system for performing imaging of cell co-cultures, may use the optical imaging system described here. A system for performing imaging of tissue sections, may use the optical imaging system described here.

While various details have been described in conjunction with the exemplary implementations outlined above, various alternatives, modifications, variations, improvements, and/or substantial equivalents, whether known or that are or may be presently unforeseen, may become apparent upon reviewing the foregoing disclosure. Accordingly. the exemplary implementations set forth above, are intended to be illustrative, not limiting.

What is claimed is:

1. An optical imaging system having a focal plane and optical elements, for imaging a biological sample, comprising:
a stage holding the sample wherein the stage is configured to continuously move the sample in at least one direction parallel to the focal plane;
at least one non-point-like light source emitting at least one wavelength;
at least one non-point-like light source disposed adjacent to a plane conjugate to the focal plane of the microscope, and
a detector which forms an image of the sample on the moving sample stage, wherein the detector has at least one row of pixels to which the image of the sample moves perpendicular during the imaging process.

2. The optical imaging system of claim 1, wherein the detector is a time delayed integration (TDI) detector.

3. The optical imaging system of claim 1, wherein the at least one non-point-like light source emits a plurality of wavelengths.

4. The optical imaging system of claim 1, wherein the at least one non-point-like light source comprises at least one of a laser, an array of lasers, an optical fiber, an array of optical fibers, a laser diode, an array of laser diodes, an LED, an array of LEDs, an incandescent lamp, an array of incandescent lamps, a gas-discharge lamp, and an array of gas-discharge lamps.

5. The optical imaging system of claim 1, wherein the at least one non-point-like light source is anamorphotically imaged onto a sample plane containing the biological sample.

6. The optical imaging system of claim 1, further comprising a downstream workflow, wherein the downstream workflow includes at least one of proteomics, genomics, and transcriptomics.

7. The optical imaging system of claim 6, further comprising a wavelength separating element, wherein the wavelength separating element separates wavelengths by diffraction, refraction, transmission or reflection.

8. The optical imaging system of claim 1, further comprising a downstream workflow, wherein the downstream workflow includes antigen retrieval of tissue, tissue lysing, padlock probe hybridization and ligation on RNA or reverse transcription of RNA, and circularization of the padlock probe and enzymatic amplification.

9. The system of claim 1, wherein the imaging system further includes a methodology which distinguishes genetic base pairs, and therefore forms at least a portion of a DNA sequencing methodology.

10. The optical imaging system of claim 7, wherein the wavelength separating element comprises at least one of a prism, a wavelength dependent turning mirror and a diffraction grating.

11. The system of claim 7, wherein the wavelength separating element comprises an adjacent angled mirror with a plurality of surfaces, wherein at least one surface is dichroic.

12. The optical imaging system of claim 1, wherein the optical elements include at least one optical element chosen from the group consisting of a light amplitude modulating element, a light phase modulating element, a light refracting element, and wherein at least one optical element is disposed in an intermediate image plane of the optical imaging system.

13. The optical imaging system of claim 10, wherein the intermediate image plane is also in image plane of the detector.

14. A DNA sequencing system using the optical imaging system of claim 1.

15. An RNA sequencing system using the optical imaging system of claim 1.

16. A system for performing spatial transcriptomics, using the optical imaging system of claim 1.

17. A system for performing spatial genomics, using the optical imaging system of claim 1.

18. A system for performing spatial proteomics, using the optical imaging system of claim 1.

19. A system for imaging of cell monolayer, using the optical imaging system of claim 1.

20. A system for performing imaging of cell co-cultures, using the optical imaging system of claim 1.

21. A system for performing imaging of tissue sections, using the optical imaging system of claim 1.
